(19) United States
(12) Patent Application Publication
(10) Pub. No.: US 2011/0106151 A1
(43) Pub. Date: May 5, 2011

(54) SQUARE LOCK SUTURE KNOT

(76) Inventors: Dennis McDevitt, Raleigh, NC (US); Vincent Novak, Longmont, CO (US)

(21) Appl. No.: 12/841,681

(22) Filed: Jul. 22, 2010

Related U.S. Application Data
(60) Provisional application No. 61/271,534, filed on Jul. 22, 2009.

(51) Int. Cl. A61B 17/04; B65H 69/04

(52) U.S. Cl. 606/228; 289/1.5

(57) ABSTRACT

A method for forming a suture knot, the method comprising:
folding a strand of suture in half;
forming two dog-ear loops at the bend of the suture strand;
folding down the two dog-ear loops so as to form two stacked loops;
connecting the loops through a suture bridge that crosses the two limbs of the strand of suture;
placing the ends of the two limbs through the stacked dog-ear loops; and
flipping the suture bridge to the opposite side of the knot body.

### REFERENCE TO PENDING PRIOR PATENT APPLICATION

This patent application claims benefit of pending prior U.S. Provisional Patent Application Ser. No. 61/271,534, filed Jul. 22, 2009 by Dennis McDevitt et al. for SQUARE LOCK SUTURE KNOT (Attorney's Docket No. INCUMED-13 PROV), which patent application is hereby incorporated herein by reference.

### FIELD OF THE INVENTION

This invention relates to surgical methods and apparatus in general, and more particularly to methods and apparatus for securing tissue with suture.

### BACKGROUND OF THE INVENTION

In arthroscopic surgery, a surgeon's fingers cannot reach the suture that runs through the opposing sides of torn soft tissue to ensure that the suture knot is tied tight and the tissues held in apposition. As such, after passing a suture strand through the soft tissue with the use of an arthroscopic suture passer, surgeons typically form a slipknot or sliding knot (extra-corporeal) and, in conjunction with a knot pusher (which acts as an extension of their finger), pushes or slides the formed knot through a portal down to the soft tissues, and through the manipulation of the suture limbs, are able to tighten the subsequently formed suture loop into a suture stitch.

FIG. 1 depicts a suture strand having limbs A and B. FIGS. 2-4 depict a simple sliding knot. The surgeon wraps the sutures in such a fashion that the body of the knot is formed on one limb A of the suture strand, while the opposing limb B runs through the body of the knot. As the surgeon pushes the knot body on limb A, he/she pulls limb B, and the formed suture loop begins to tighten around the tissue to be fixed. As the suture loop tightens, the knot body in limb A tightens around limb B, thereby forming a knotted suture loop. The surgeon then places several more sliding knots on top of each other to prevent the knot from slipping or loosening during the post-operative healing period.

Over the past fifteen years, there has been great interest in developing new arthroscopic knots. Development has focused on slipknots versus a locking knot (i.e., a square knot) because surgeons are not able to adequately slide or push a locking knot through a portal and then down to the interior repair site. The locking knot would often “lock up” before the suture loop tightened around the soft tissue, requiring the removal of the suture and another new attempt by the surgeon. As a result, surgeons investigated slipknots that resist slipping under the normal biomechanical loads that the repaired tissue would experience. This led to a variety of slipknots that increased the bulk of the knot body, the idea being that increased bends in the suture would increase the friction between the suture limbs, thereby building resistance to slippage. However, this often led to knots with high and/or large profiles within a joint that raised the risk of new soft tissue injury coming from the knot itself.

Recently, the pendulum has swung back from the development of complex sliding knots to the more common and simplest knots, the half-hitches or surgeon knots shown in FIGS. 1-4. Surgeons have felt that they can more adequately control the size of the knot, and even though it is well understood in the field that these knots may be prone to slippage if not adequately tightened, surgeons have learned to place multiple stitches to share the biomechanical load on the tissue repair.

Thus there is a need for a new and improved knot for arthroscopic suturing.

### SUMMARY OF THE INVENTION

The present invention comprises the provision and use of a new and improved knot for arthroscopic suturing. As will hereinafter be discussed, the present invention provides a novel knot (i.e., the “square lock suture knot”) which presents a sliding knot that, once slid into place and the suture loop tightened, can be turned into a locking knot.

In one preferred form of the invention, there is provided a method for forming a suture knot, the method comprising:

folding a strand of suture in half;

forming two dog-ear loops at the bend of the suture strand;

folding down the two dog-ear loops so as to form two stacked loops;

connecting the loops through a suture bridge that crosses the two limbs of the strand of suture;

placing the ends of the two limbs through the stacked dog-ear loops; and

flipping the suture bridge to the opposite side of the knot body.

In another preferred form of the invention, there is provided a method for securing a first piece of tissue to a second piece of tissue, the method comprising:

passing a strand of suture through the first piece of tissue and the second piece of tissue;

folding a strand of suture in half;

forming two dog-ear loops at the bend of the suture strand;

folding down the two dog-ear loops so as to form two stacked loops;

connecting the loops through a suture bridge that crosses the two limbs of the strand of suture;

placing the ends of the two limbs through the stacked dog-ear loops; and

flipping the suture bridge to the opposite side of the knot body.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 illustrates a suture strand having two limbs A and B;

FIGS. 2-4 illustrate a simple sliding knot;

FIGS. 5-10 illustrate a single strand of suture formed into a suture knot in accordance with the present invention;

FIGS. 11 and 12 illustrate a suture with a two-limb knot;

FIGS. 13-15 illustrate a single-limb knot; and

FIG. 16 illustrates the fixing of limb A (i.e., the limb that forms the body of the knot) behind the knot body on limb B after forming the knot body itself.

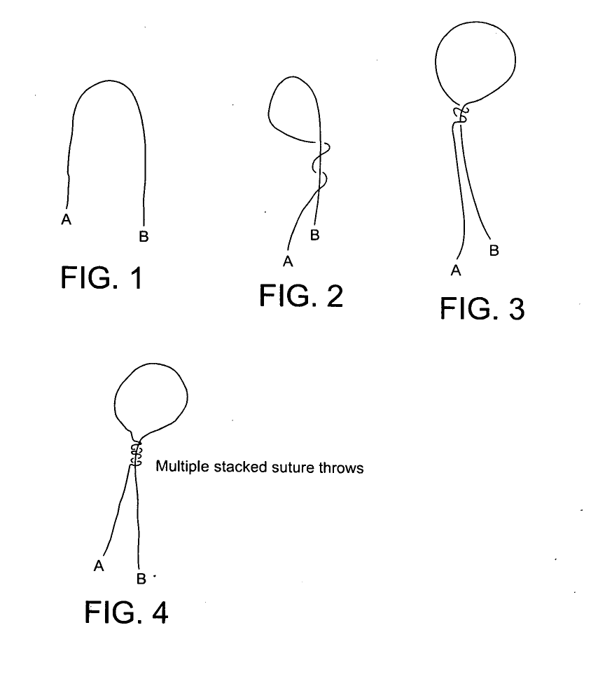

### DETAILED DESCRIPTION OF THE PREFERRED EMBODIMENTS

The present invention comprises the provision and use of a novel knot (i.e., the “square lock suture knot”) that, once slid into place and the suture loop tightened, can be turned into a locking knot.

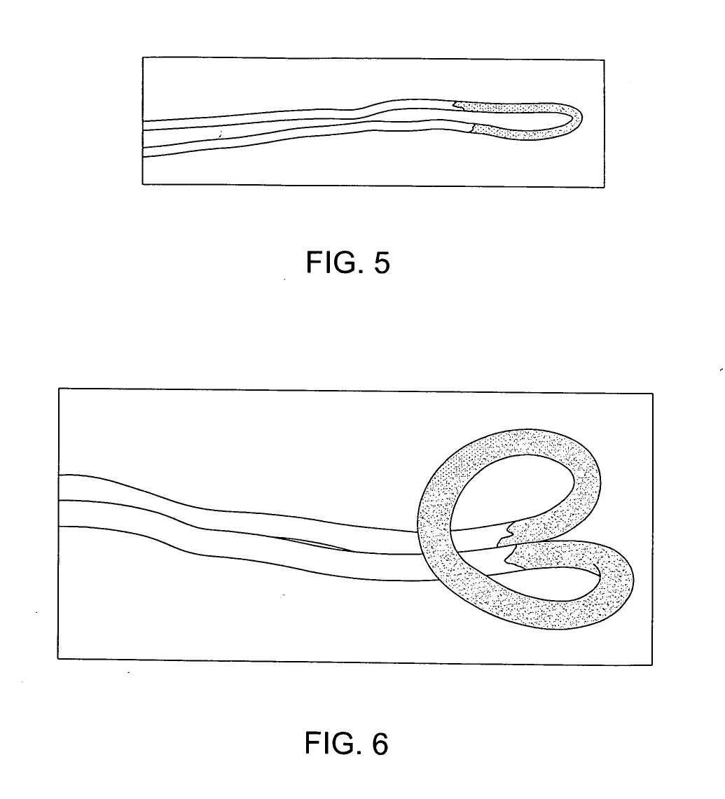

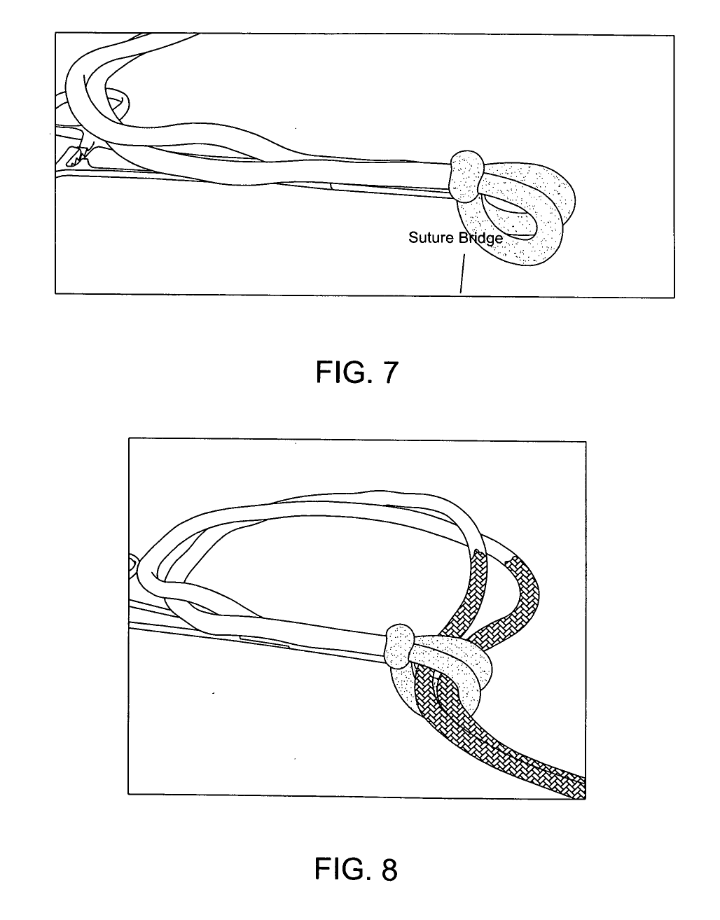

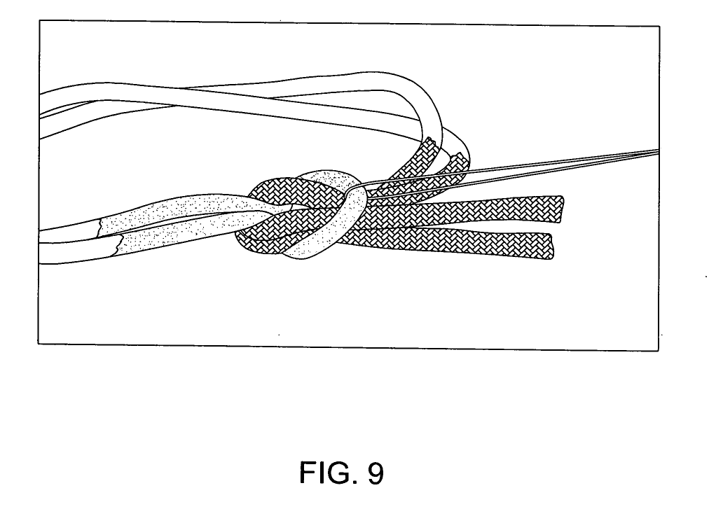

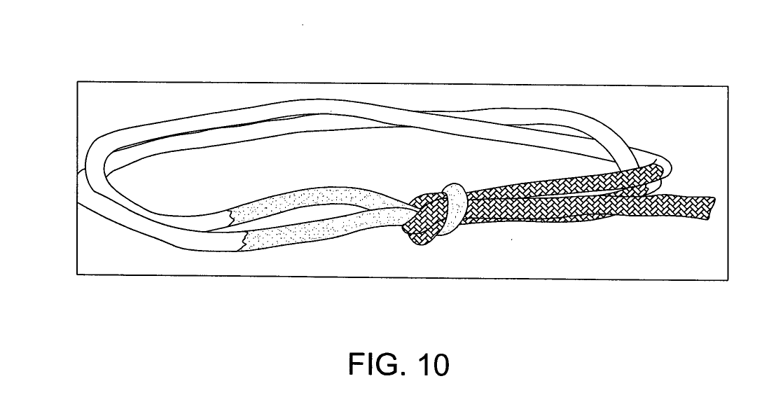

More particularly, FIG. 5 shows a single strand of suture (schematically shown using a length of rope rather than suture for improved clarity of viewing) folded in half. FIG. 6 shows the formation of two dog-ear loops at the bend of the suture strand (shown colored in red). FIG. 7 shows the dog-ear loops folded down, forming two stacked loops. The loops are connected through a “suture bridge” that crosses the two limbs of the single strand. FIG. 8 shows the ends of the two limbs (shown colored in blue) being placed through the stacked dog-ear loops. FIG. 9 shows the suture bridge being flipped to the opposite side of the knot body. FIG. 10 shows a locked knot, i.e., the locked “square lock suture knot”.

The locking knot described above and illustrated in FIGS. 5-10 show the knotting of both (two) limbs of a single strand.

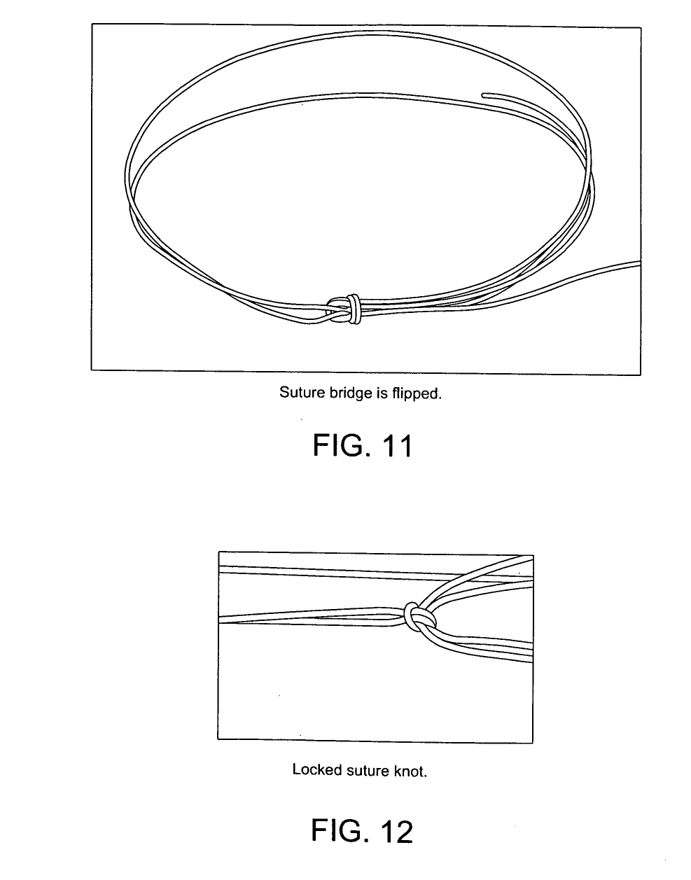

FIGS. 11 and 12 show a #2 suture with a two-limb knot.

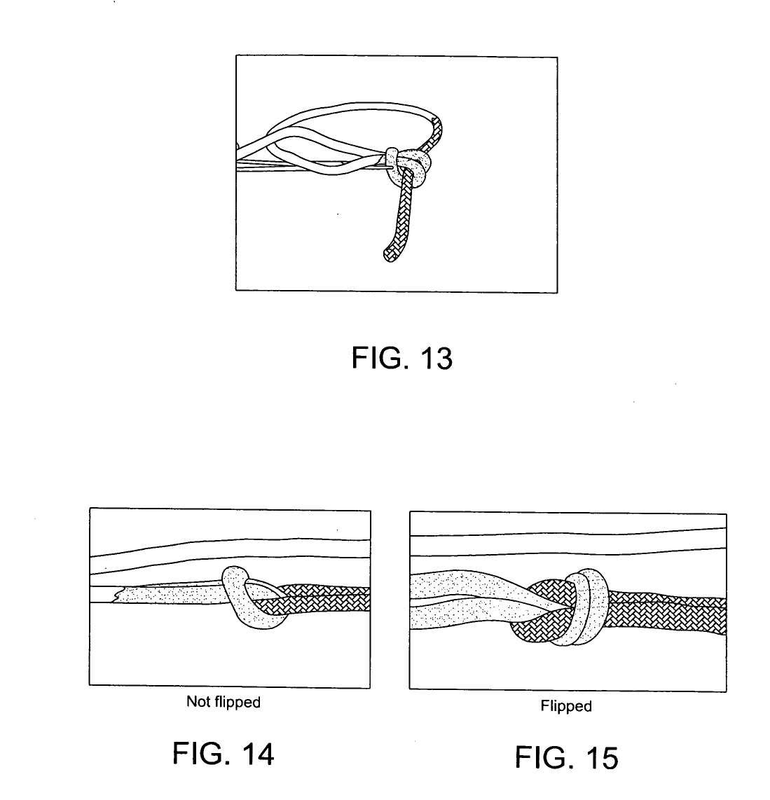

A single-limb knot can also be provided and used, and is shown illustrated in FIGS. 13-15.

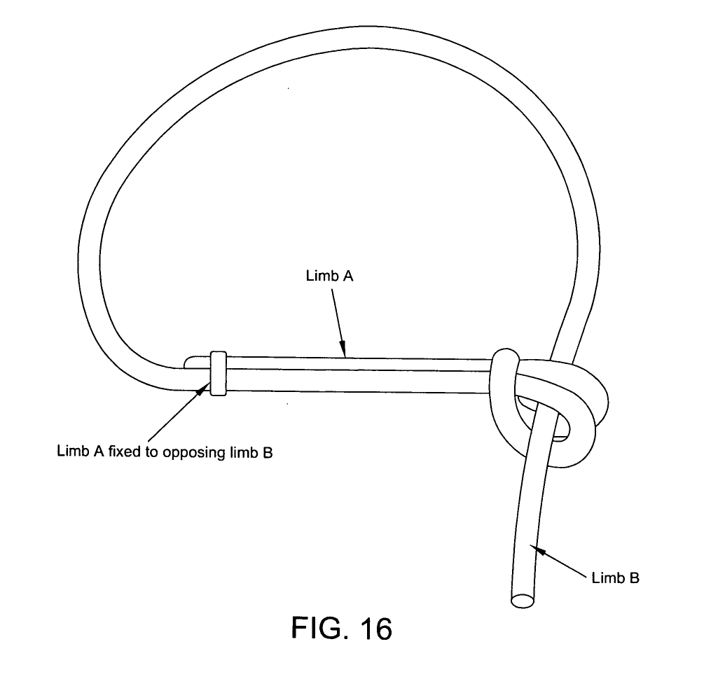

With regard to a single-limb knot of the sort illustrated in FIGS. 13-15, FIG. 16 shows the importance of fixing limb A (i.e., the limb that forms the body of the knot) behind the knot body on limb B (after forming the knot body itself). This prevents a single-limb, locked knot from twisting under biomechanical tensile loads, and forcing the suture bridge to its pre-flipped position, which may lead to subsequent slippage and early knot failure.

Ultimate tensile testing (UTS) has demonstrated that the traditional sliding knot employed by most surgeons failed at approximately 30 pounds of tension (with minimal knot slippage). The disclosed single-limb square lock suture knot of the present invention demonstrated similar results. The two-limb square lock suture knot exceeded 50 pounds of tensile load without knot slippage.

### MODIFICATIONS

It will be understood that many additional changes in the details, materials, steps and arrangements of parts, which have been herein described and illustrated in order to explain the nature of the invention, may be made by those skilled in the art while remaining within the principles and scope of the present invention.

What is claimed is:

1. A method for forming a suture knot, the method comprising:
folding a strand of suture in half;
forming two dog-ear loops at the bend of the suture strand;
folding down the two dog-ear loops so as to form two stacked loops;
connecting the loops through a suture bridge that crosses the two limbs of the strand of suture;
placing the ends of the two limbs through the stacked dog-ear loops; and
flipping the suture bridge to the opposite side of the knot body.

2. A method for securing a first piece of tissue to a second piece of tissue, the method comprising:
passing a strand of suture through the first piece of tissue and the second piece of tissue;
folding a strand of suture in half;
forming two dog-ear loops at the bend of the suture strand;
folding down the two dog-ear loops so as to form two stacked loops;
connecting the loops through a suture bridge that crosses the two limbs of the strand of suture;
placing the ends of the two limbs through the stacked dog-ear loops; and
flipping the suture bridge to the opposite side of the knot body.
